(19) United States
(12) Patent Application Publication
(10) Pub. No.: US 2011/0046489 A1
(43) Pub. Date: Feb. 24, 2011

(54) SYSTEMS AND METHODS EMPLOYING GIANT STOKES SHIFT

(75) Inventors: Hirak Kumar PATRA, Midnapore (IN); Anjan Kumar Dasgupta, Kolkata (IN)

Correspondence Address: PILLSBURY WINTHROP SHAW PITTMAN, LLP, P.O. BOX 10500, MCLEAN, VA, 22102, US

(73) Assignee: UNIVERSITY OF CALCUTTA, Kolkata (IN)

(21) Appl. No.: 12/628,721

(22) Filed: Dec. 1, 2009

(30) Foreign Application Priority Data
Aug. 18, 2009 (IN) 1076/KOL/2009

(51) Int. Cl. A61B 6/00; G01N 21/84; G01N 21/64; G01J 5/02; G01J 5/10

(52) U.S. Cl. 600/473; 250/330; 250/339.07

(57) ABSTRACT

A method comprising exposing a sample comprising water to near infrared (NIR) light and detecting the presence of one or more objects by measuring a Stokes shift in the emission spectra in the near infrared.

### CROSS-REFERENCE TO RELATED APPLICATION

The present application claims the benefit of Indian Patent Application No. 1076/KOL/2009, filed Aug. 18, 2009, which is hereby incorporated by reference in its entirety.

### BACKGROUND

Near-infrared fluorescence (NIRF) is an emerging branch within the field of fluorescence spectroscopy. In the biological context, the advantages of NIR over UV and visible region fluorescence include a lower background signal from biological samples and a deeper penetration of the radiation into biomatrices. Tissues, proteins or other biomarkers fluoresce naturally due to the fluorescence of the amino acids like tryptophan, Tyrosine, and Phenyl Alanine. Further, there is always background interference in the visible region of the electromagnetic spectrum. This background signal is a barrier in the detection of biological samples when fluorescence labels in the visible region are used. In the NIR region, however, this background is absent. Furthermore, biological samples have less light scattering of NIR radiation, as Rayleigh scattering shows a 1/λ⁴ dependence. That is, the longer the wavelength, the lower the Rayleigh scattering.

The deep penetration of NIR is a factor in the NIR based spectroscopic labels and in vivo imaging. The penetration of the NIR radiation can reach 2-5 cm into a sample. Thus, the NIR region often is referred to as the “Biological Window”.

NIR optical imaging may be useful in disease detection and staging, drug development, and treatment assessment. There is, however, a problem. That is the optical properties of water in the NIR region is poorly understood. The so called auto-fluorescence of tissues on which clinical detections like Alzheimer disease are made may actually be a reflection of diversity of the water structure and diverse ordering of water molecules at different biological interfaces, including the sub-cellular region.

In current practice, nanoparticle based bio-labelling techniques and nanoparticle mediated drug delivery for therapeutic reasons are looked upon as independent problems. In the first case, dyes (with certain absorption cross section in the electromagnetic spectrum) are being used. In the second case, drugs are conjugated to a nanosurface. The dyes tagged for labelling often have known level of cytotoxicity and are well known as biohazards molecules or reagents. The optically promising nanomaterials on the other hand, like quantum dots, are well known for their high level of toxicity. Further, conjugating drugs to nanomaterials can result with interference with the functional ability of the drug molecules. Thus, rendering the drug less effective or ineffective.

### BRIEF DESCRIPTION OF THE FIGURES

FIG. 1 shows the near infrared (NIR) fluorescence of water.

FIG. 2 is a fluorescent spectra of an embodiment illustrating (a) the NIR response of water in the presence of various size citrate capped gold nanoparticles, and (b) an enlargement for the smaller particles whose fluorescence are close together.

FIG. 3 shows the surface conjugation (morphology) effect on fluorescence of an embodiment.

FIG. 4 shows the thermal effect of NIR fluorescence of an embodiment.

FIG. 5 illustrates an embodiment with latex beads.

FIG. 6 illustrates a time profile of the NIR fluorescence with platelets in absence (lighter line) and presence (darker line) of an agonist (collagen).

FIG. 7 illustrates NIRF specificity to water (shown with the darker line in each panel) relative to other solvents including: (a) ethanol, (b) DMSO, (c) methanol (MeOH), and (d) chloroform. Water shows a predominant NIRF in each case

FIG. 8 illustrates NIRF diversity to different water/particle interfaces including: (a) pure water, (b) bacteria, (c) gold nanospheres, and (d) platelets.

FIG. 9 illustrates the hydrodynamic size of nanoparticles used in the Examples, including: (a) 20 nm gold nanopartilces (GNP), (b) 30 nm GNP, (c) 40 nm, (d) 150 nm GNP, GNP, (e) 30 nm GNP coated with arginine, (f) 40 nm GNP coated with arginine, (g) 150 nm GNP coated with arginine, and (h) 60 nm latex.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

An embodiment relates to a method comprising exposing a sample comprising water to near infrared (NIR) light and detecting the presence of one or more objects by measuring a Stokes shift in the emission spectra in the NIR. In one aspect, the detected object is not conjugated with an IR label. In another aspect, the objects range in size from 5 nm to 10 micron. In another aspect, the method is sensitive to object size. In another aspect, the method integrates detection of nanoparticles and drug targeting without conjugated NIR dyes. In another aspect, the method further comprises detecting a change in shape or change in surface property. In another aspect, the method further comprises evaluating the progress of a disease. In another aspect, the excitation wavelength is approximately 640 nm and wherein the emission spectra has a peak in a range of approximately 850-1000 nm. In another aspect, the objects are capped with arginine. In another aspect, the objects are biological and further comprising modifying the object with an agonist.

An embodiment relates to a system comprising a light source configured to emit light onto a sample comprising water and a detector configured to detect a Stokes shift in an emission spectra in the NIR. In one aspect, the light is emitted at a wavelength is approximately 640 nm and wherein an the emission spectra has a peak in a range of approximately 850-1000 nm. In another aspect, the system further comprises a sample excited by the light, the sample comprising particles. In another aspect, the particles are gold or latex beads. In another aspect, the objects are biological.

Another embodiment relates to a method comprising supplying objects that range in size from 5 nm to 10 micron to a sample to be imaged, the sample comprising water, exposing the sample to near infrared (NIR) light and obtaining an image of the sample. In one aspect, obtaining an image comprises special mapping. In one aspect, obtaining an image comprises determining the structure of a cell. In one aspect, the particles are gold or latex beads.

Another embodiment relates to a method comprising using the system for an application comprising detecting nano-scale or micro-scale objects in an aqueous sample or imaging a biostructure or detecting particulate pollution in a water sample; or detecting a drug conjugated nanoparticle.

A near infrared (NIR) spectrum region based method has been developed. Using this method it is possible to detect particulate matters whose size can range from nano-scale to micron scale. The methodology does not require any specific NIR probe yet can produce material and size specific diagnostic signatures. The approach is not restricted to nano size objects only. The proposed approach can integrate detection of nanoparticle location and drug targeting without special need to conjugate NIR dyes.

The NIR region is known for its high tissue permeability and is less hazardous than UV. The clinical application of the proposed spectroscopic technique is thus evident. Further, rapid detection of nano to micron scale objects can be possible by this method. For example, one can detect one of the precious and smaller blood components like platelets. Additionally, with the methods described in more detail below, one can detect a shape change of a platelet or other nano-structure. This may be accomplished, for example, when the shape change is induced by agonists.

Such detection may have immense value in the prognosis of platelet related diseases, for example cardiovascular diseases. Similarly one can use the technique to detect bacteria. Since the method is sensitive to nanoscale objects, it can even be used to detect the uptake of nano scale objects by a micro-object like bacteria.

Applicants have discovered a previously unknown NIR fluorescence associated with the aqueous interface of objects in the nanometer to micrometer scale. A giant Stokes shift is observed in the NIR regime for both excitation and emission. A “giant stokes shift” means a shift of approximately 200-300 nm. Typical near-infrared dyes have a stokes shift of approximately 30 nm. Large, 100-150 nm Stokes shift dyes have been reported.

The intensity and the shift of the discovered aqueous NIR fluorescence are a function of the object size and the constituting object material. Numerous applications of the observed Stokes shift are possible as NIR regions are free of interferences from stray visible light while also having high penetration in complex media, cells and tissues.

Water has a versatile structure and it is precisely the dynamicity of interactions between water molecules that makes biological systems manifest their structural and functional diversity. It is the variation in water structure that reflects the different molecular and cellular interactions and those at the tissue level, as 90% of biological system contains water. Outside the biological world, the behavior of many colloids also depend on the water interface. So cells with characteristic ordering of water molecules in them should have NIR fluorescence.

The embodiments of the method may be useful for tracking nanoparticles localized in a subcellular domain, especially nanoparticles loaded with drugs. NIR probes/labels (often cyto-toxic) are not needed to perform the various methods. Further, the methods may be exploited to determine the presence and/or absence of particulate matters in water. The particulate matter may include, but are not limited to, nanoscale objects (nanoparticles), microscale objects (bacteria or platelets) and inert objects (latex beads).

The NIR region is of keen current interest. The embodiments herein relate to exploiting the NIR spectra of materials comprising water, wherein the NIR spectra has a strong peak in the region of 900-1000 nm for excitation at 640 nm. The NIR spectra of water has two major peaks, 900 nm and 1000 nm. Out of the two peaks, one showed a minimal shift of maxima position (˜900 nm) when the excitation was varied. The other peak (˜1000 nm) is relatively sensitive to variation of the excitation level. For pure water, this peak is present but highly attenuated. In the presence of objects in aqueous medium, however, the peak intensity increased. The level of increase was found to be object size and concentration dependent. That is, it is possible to characterize a NIR fluorescence that is amplified on the object water interface in a manner that is a function of the object dimensions.

The peak is strong enough to respond to minor changes in temperature. The fact that water structure and its diversity plays a major role in dictating the observed behavior is supported by the fact that colloidal gold particles shows a discontinuous thermally induced change when the temperature is brought down from >4° C. to <4° C. Such a discontinuity is a typical signature of water. The temperature effect was reversed when the colloidal solution of citrate capped gold was further conjugated with arginine molecule. The thermal behavior of such conjugation as reflected by the NIR 900 nm Stokes line is complementary to that exhibited by the bare gold nanoparticle. In other words, the proposed methods may be a powerful tool to explore surface conjugation.

The disclosed methods can be applied in multiple domains ranging from medicine to manufacturing. For example, the NIR method is capable of detecting micro-scale objects, such as organelles (bacteria and platelets). The methods can measure specific responses of such organelles to various stimulants (e.g. agonists) or anatagonists (inhibitors) that lead to the organelle's shape or size change with respect to time. The method does not require specialized chemical substances (NIR dyes), the toxicity of NIR dyes often being a problem. The NIR region being transparent to tissues, the method is amenable to bio-localization studies based on variation in water structures in different subcellular locations. As water is ubiquitously present in biological systems and ordering of water will vary in normal and abnormal tissues; the proposed water based NIR detection system may be able to discriminate between normal and abnormal tissues. In addition to biological systems, the method can be useful in detecting particulate matter (of different dimensions) in water. Thus, the methods can be used to determine water quality via particulate pollution detection measurement.

Upon excitation in the 640 nm region, water fluorescence in the wavelength region 900-1000 nm is amplified several folds depending on the microscale or nanoscale matter (or objects) present in the aqueous environment. Water based NIR spectroscopy or NIR imaging can be performed without need of any specific NIR dye and for objects in nano or micro scale placed in a complex medium. As water is ubiquitously present in every realm of bio-systems, a technique that reflects the diversity of local ordering of water structure has potential applications in several domains. As many of the NIR dyes are known for their biohazardas properties, label free NIR imaging technique represents a paradigm shift.

### EXAMPLES

### NIRF Acquisition and Imaging System

An embodiment relates to a system comprising a light source configured to emit light onto a sample comprising water; a detector configured to detect a shift in an emission spectra in the NIR; and an imager configured to obtain an image of the sample.

The imaging system comprises a light source with a monochromator. In one embodiment, the monochromator is set at 640 nm. Monochromatic light is directed to sample including microscopic objects. The fluorescence is detected in an emission window around 900 nm. The system can detect objects having a differential aqueous interface. Variations in the size of particles and other properties (e.g. surface properties) and variations of the aqueous interface can be imaged through an IR enabled camera. As the detection system captures the variations in water structures at object interfaces, it is possible to characterize biological cell differential imaging profiles of any particular material (for example a drug) contained in a given cellular compartment. A nanoparticle contained in a cell can, in principle, be imaged using this system. Notably, this technique does not require any special fluorescence tag (or NIR tag) as the principle is based on the properties of water.

### Acquisition of NIRF Spectra:

The measurement of the NIR fluorescence was performed using an NIR compatible Quantum PTI spectrometer (PTI USA) with a NIR interface. The interface was equipped with a high intensity 75 watt xenon light source, a high sensitivity TE-cooled InGaAs detector, a lock in amplifier and a chopper for noise suppression and an additional emission mono with a 600 groove grating blazed at 1.2 microns. The data was acquired and stored through FeliX32 software (provided along with the instrument by PTI, USA). The software package included 32-bit fluorescent analysis. A 620 nm cut-off filter was used at the emission channel before the NIR detector for the prevention of the entry of stray light at the NIR region. A very weak NIR signal in water was observed to be amplified in a dimension/size and surface dependent process.

### Synthesis of Nanoparticles:

The gold nanoparticles were prepared by the standard method of Turkevich and Frens with minor modifications. Continuously stirring, an aqueous solution of HAuCl₄ (2.5 μM, 25 mL) was brought to boiling condition at 100° C. and freshly prepared trisodium citrate solution (38.8 mM) was added quickly with carrying concentrations depending on the requirement of the object size. A change in solution color from pale yellow to deep red resulted in the case of smaller nanoparticles. On generating the persistent color, the temperature was brought down to room temperature and the colloidal solution stirred for an additional 5 min. In the case of arginine conjugated nanoparticle synthesis, a final 2-10 μM L-Arginine was prepared just before the addition of the citrate.

### Optical Measurements

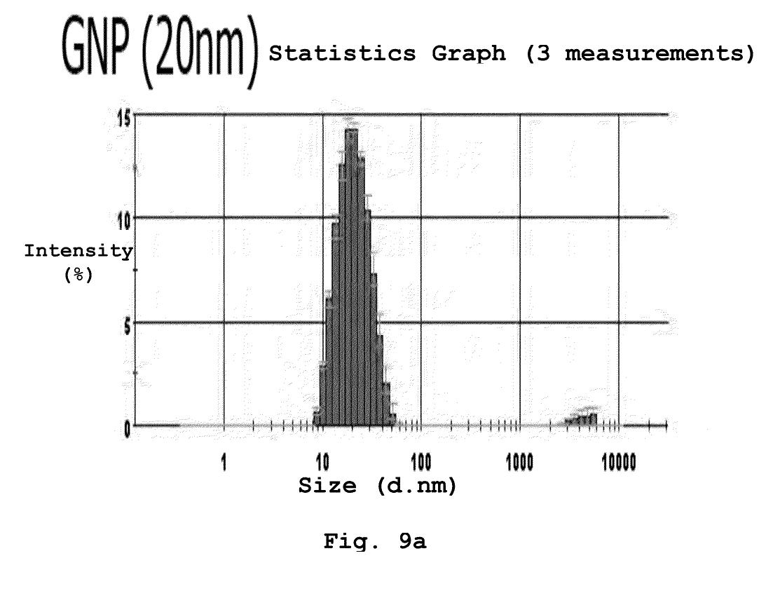

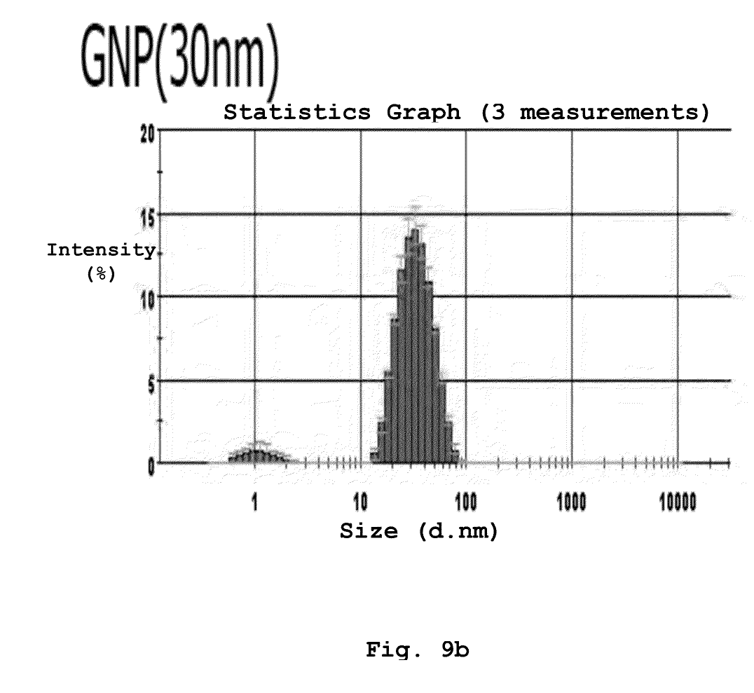

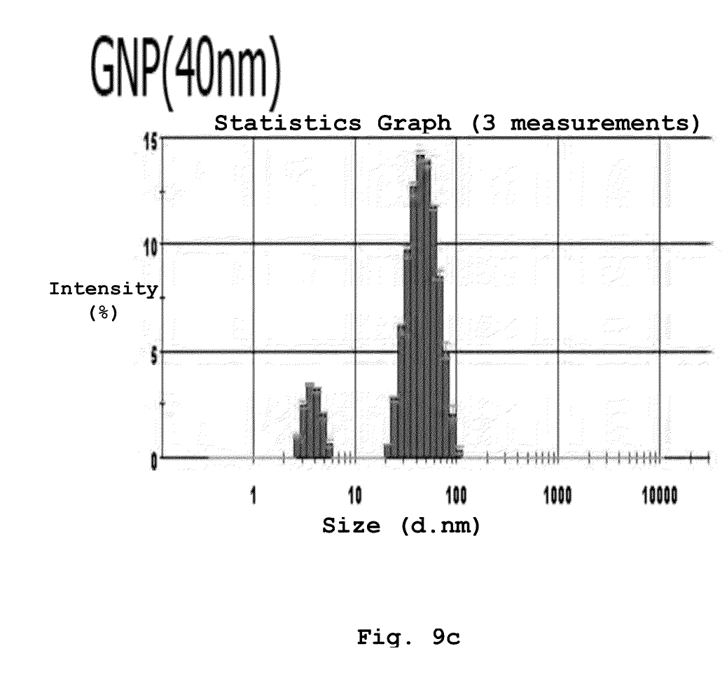

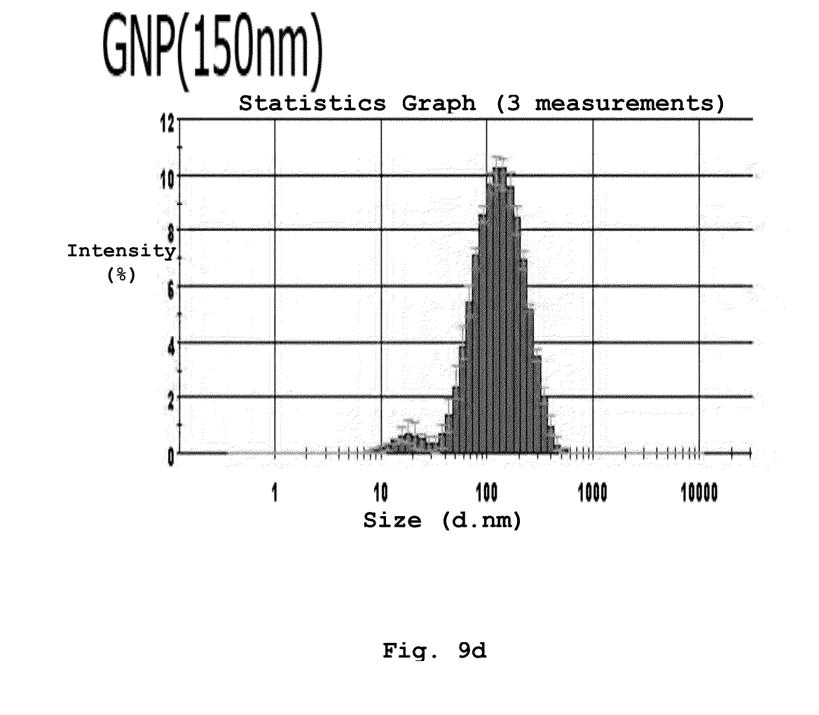

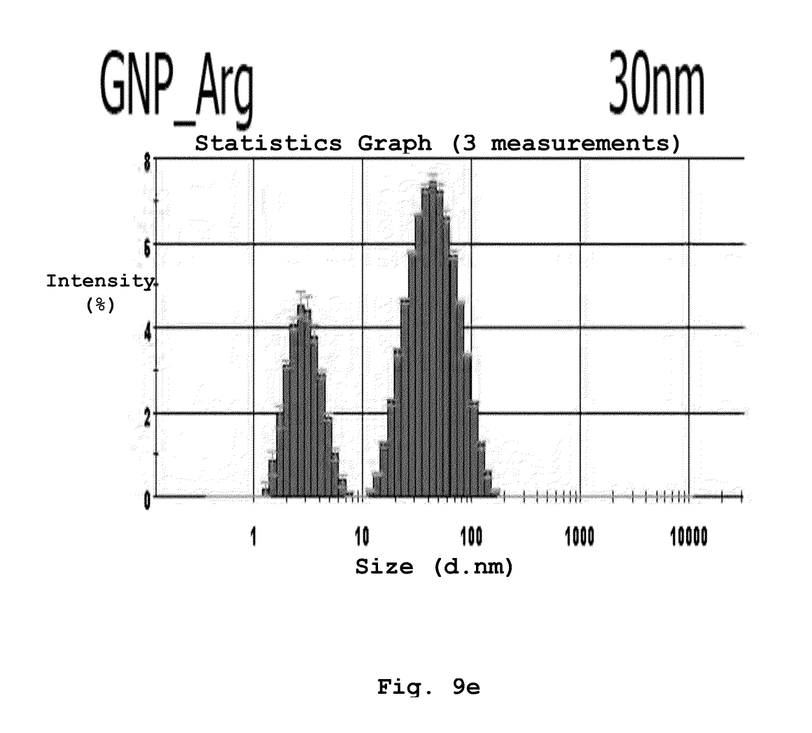

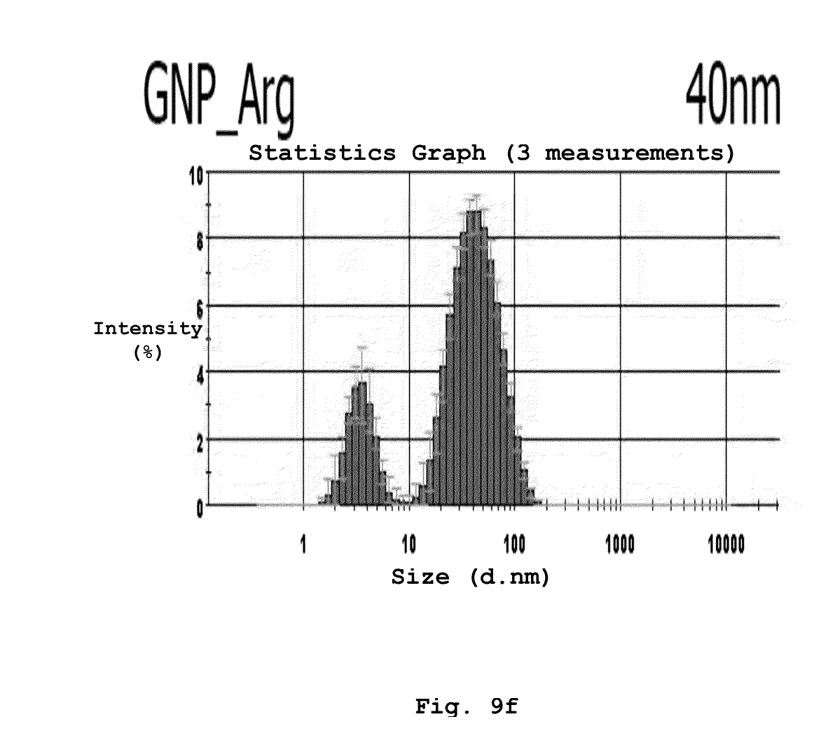

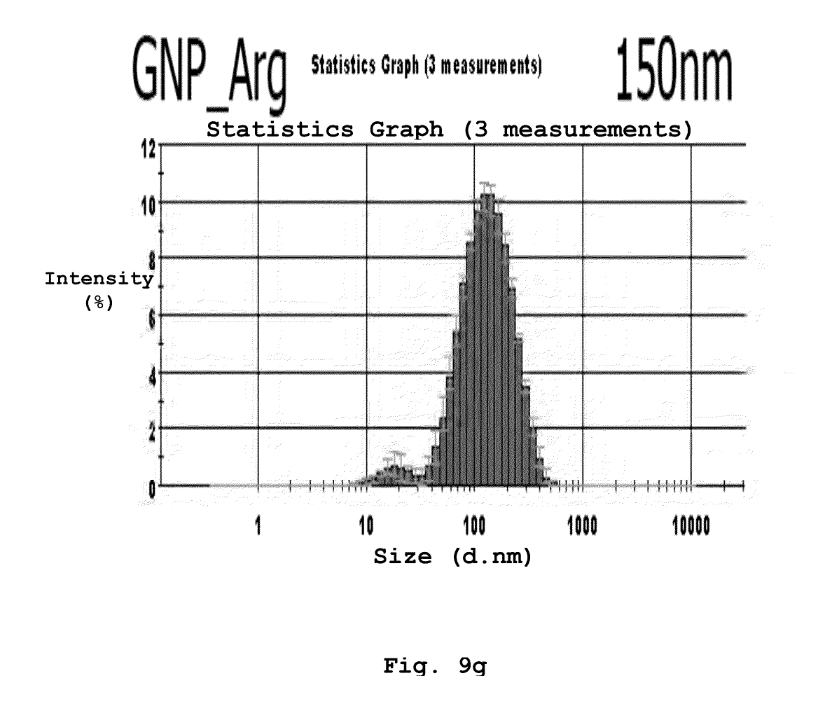

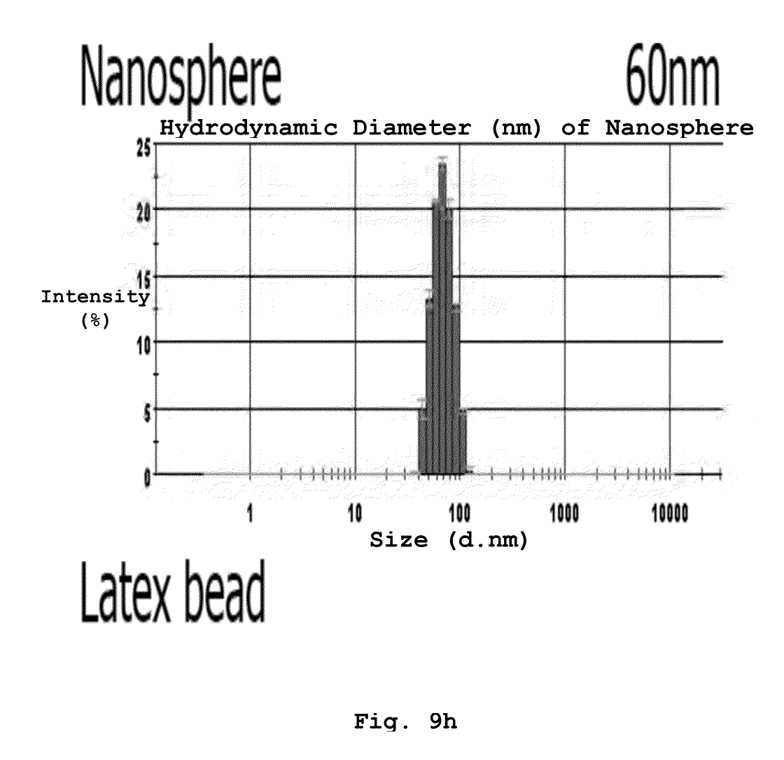

The hydrodynamic size was measured using a NanoZS (Malvern, UK). The same instrument was used to measure the C potential of the nanoparticles in colloidal suspension. The device measures the velocity of the fluid. Each particle is the average statistics of 3 sets and each set is the average of 20 measurements. The hydrodynamic size of all the nanoparticles used in this study are shown in FIG. 9.

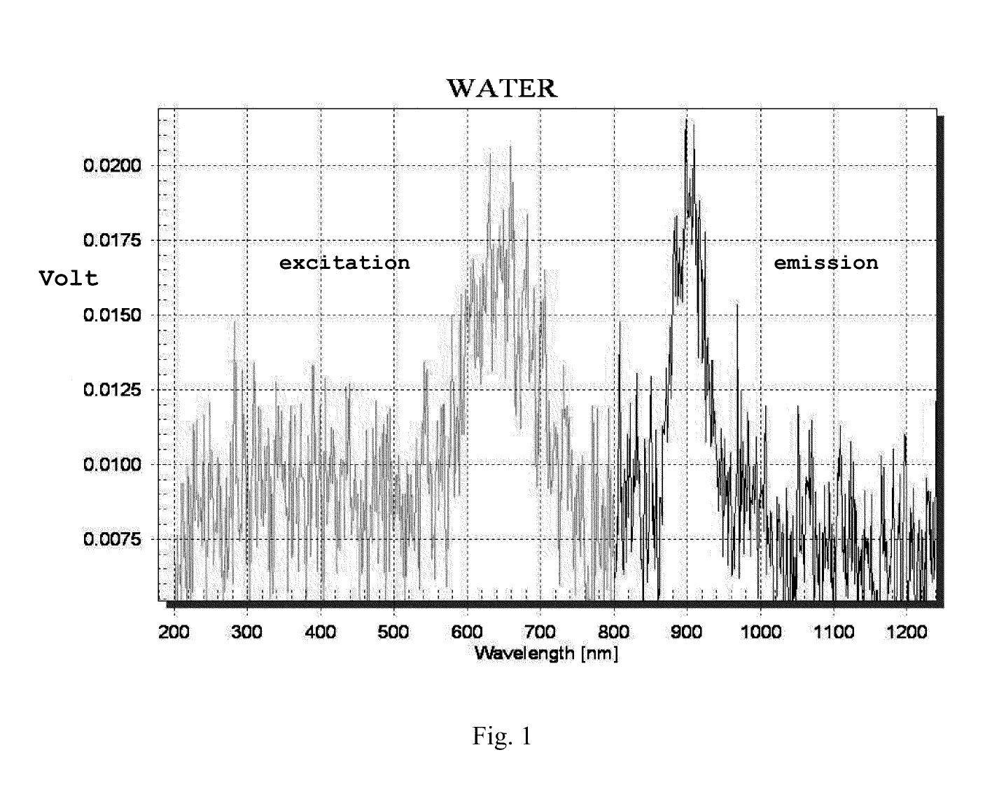

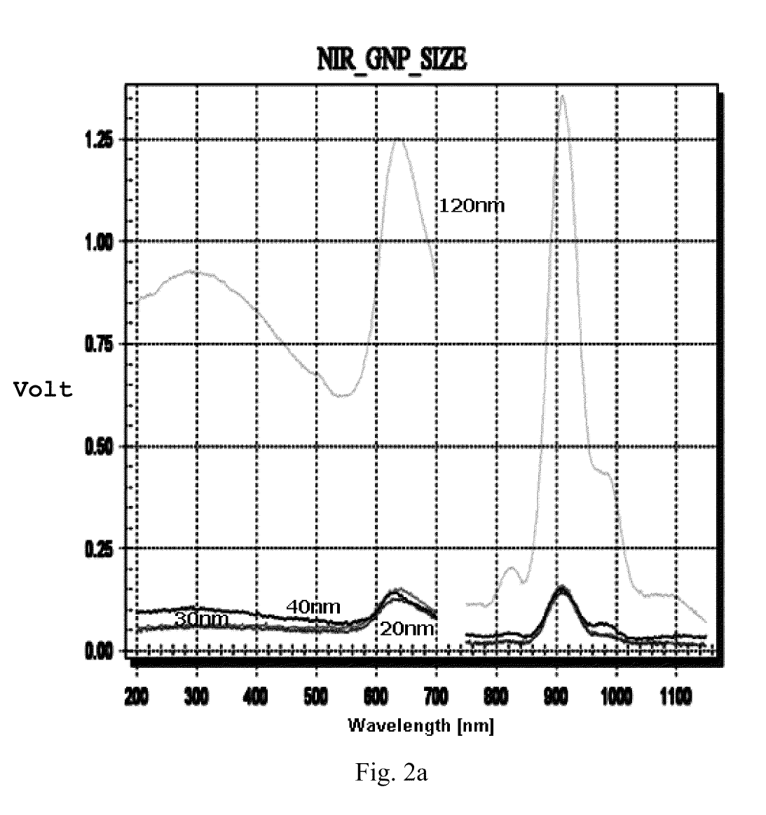

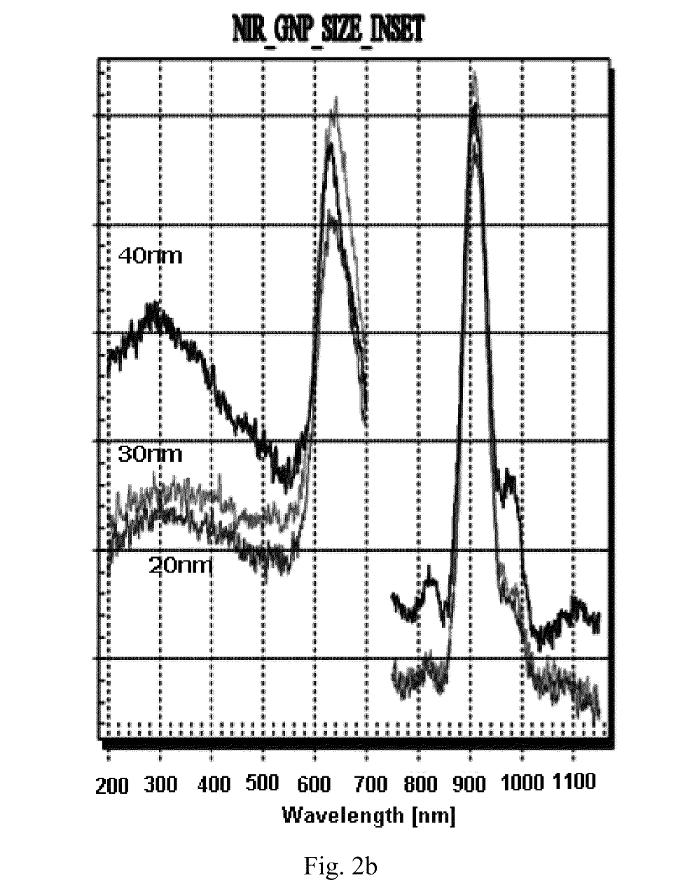

FIG. 1 illustrates a weak signal in pure water. FIG. 2 illustrates that the signal is amplified several fold in presence of nanoscale objects. Further, the intensity for particles of the same or similar materials shows a monotonic increase with size. In this example, the NIR fluorescence response as a function of size for citrate capped gold nanoparticles was measured. When nanoparticles with sizes 20 nm, 30 nm, 40 nm, and 120 nm are considered, one obtains a monotonic size dependence of fluorescence. FIG. 2(b) is an enlargement of a portion of FIG. 2(a) for the smaller particles whose fluorescence are close together. Comparison of FIG. 2 with FIG. 1 shows that the intensity axis is several fold higher than for water without particles.

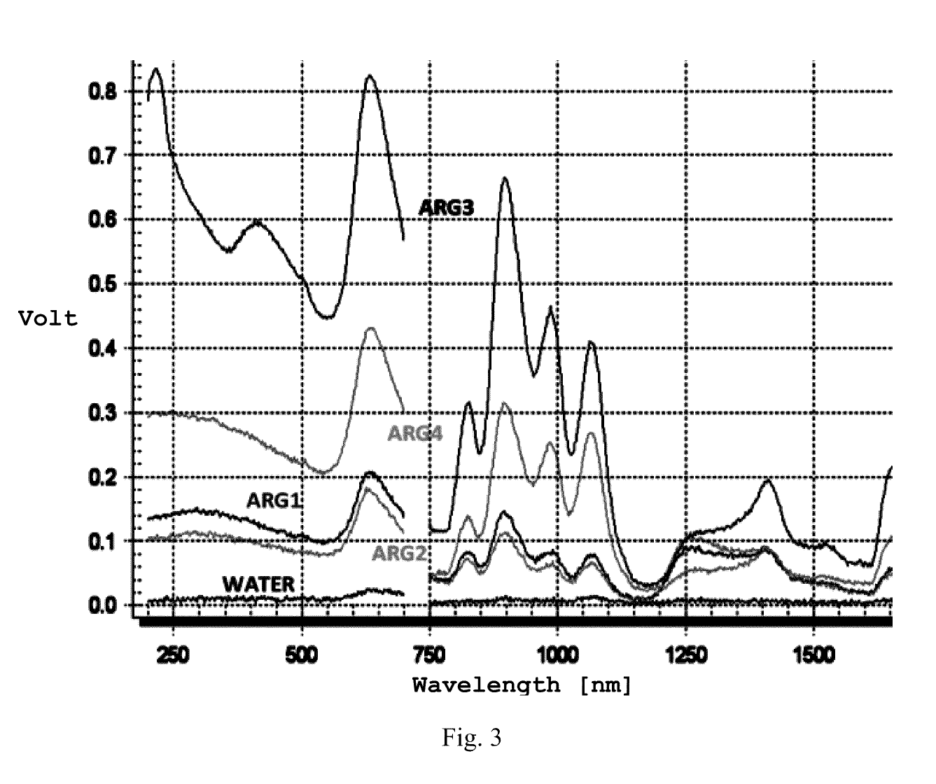

FIG. 3 shows that surface perturbation with coating agents that produce different nanosurface properties is detectable. For example, coating/conjugating with arginine shows a diverse amplification of NIR fluorescence intensity. The NIR fluorescence spectrum of arginine coated gold nanoparticles of different sizes is illustrated in FIG. 3. The curves labelled ARG1 (41 nm), ARG2 (18.89 nm), ARG3 (14.15 nm) and ARG4 (13.5 nm) respectively represents gold nanoparticles capped different arginine concentrations (2, 4, 6, and 8 uM of arginine per 250 uM of atomic gold). The spectrum labeled water represents the baseline.

The excitation and emission spectra are both illustrated. The excitation maxima in each case (including water) is seen at 640 nm. The variation in spectra represents the sensitivity of the NIR emission with respect to different types of surface conjugation. Surface conjugation varies both shape and size of nanoparticles as well as the surface hydrophobicity. That is, arginine conjugation changes the nanosurface morphology of the particles.

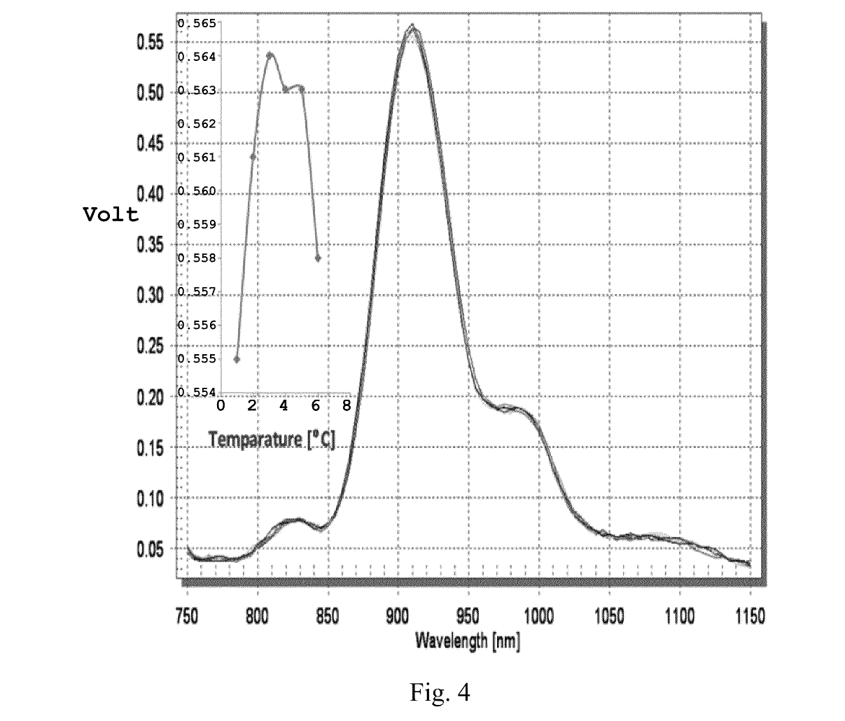

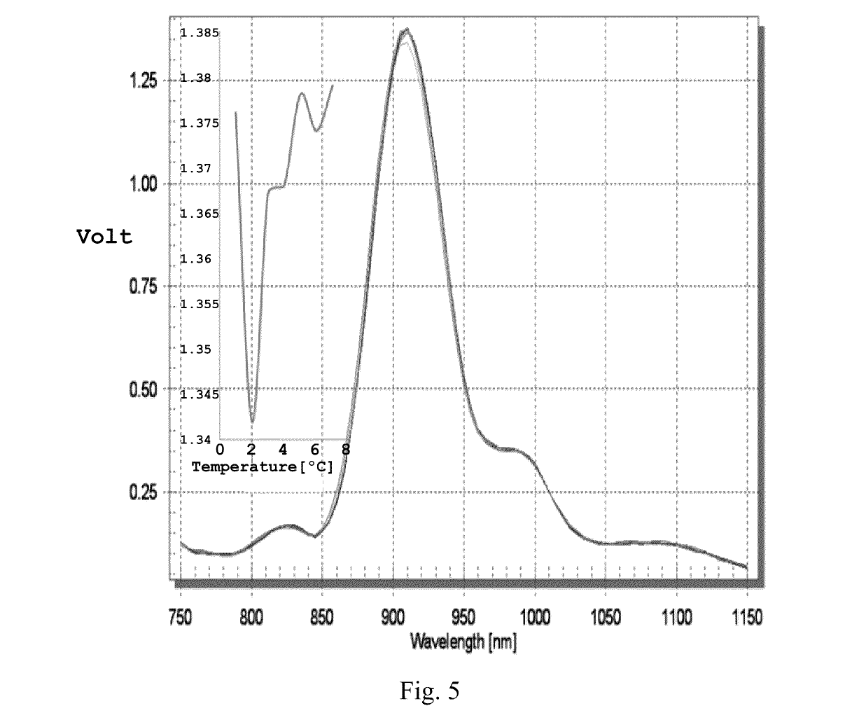

The fact that the water interface is a leading cause of the observed effect is illustrated in FIGS. 4 & 5. FIGS. 4 & 5 illustrate that at 4° C. there is a discontinuous change in the NIR fluorescence intensity. FIG. 4 illustrates the temperature effect using gold nanoparticles while FIG. 5 illustrates the temperature effect using latex beads (60 nm). This type of discontinuity is associated with the water structure. NIR fluorescence was induce with an excitation at 640 nm. Comparison of FIGS. 4 & 5 further illustrates that the thermal profiles are material-property dependent. This is evident as the surface bound water has a different degree of ordering depending on the particle surface. Further, the profile is highly sensitive to nature of surface conjugation of the nanoparticle (citrate capped nanoparticles showing a different behavior).

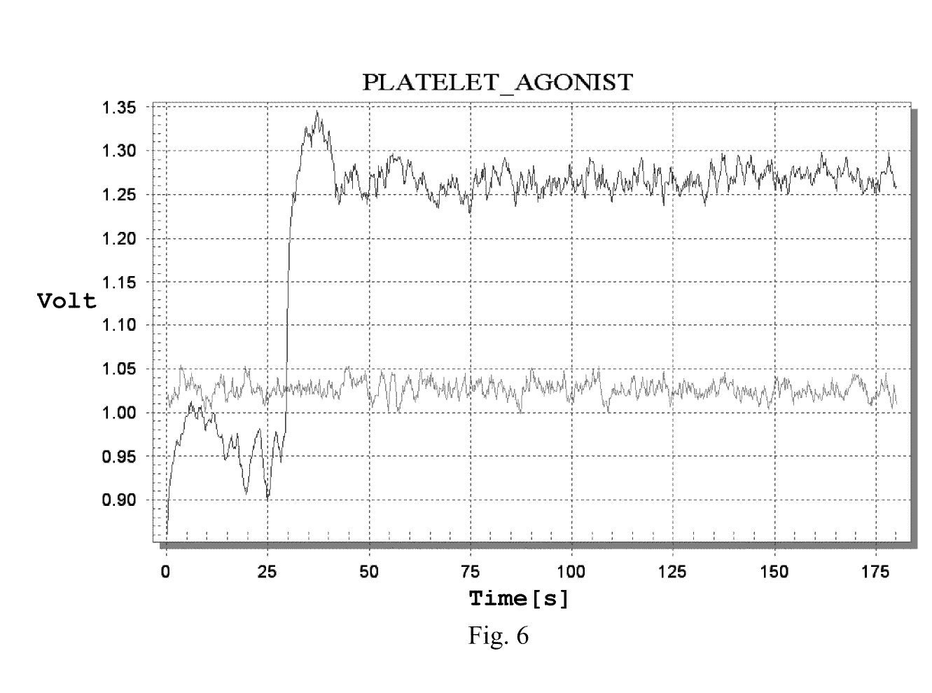

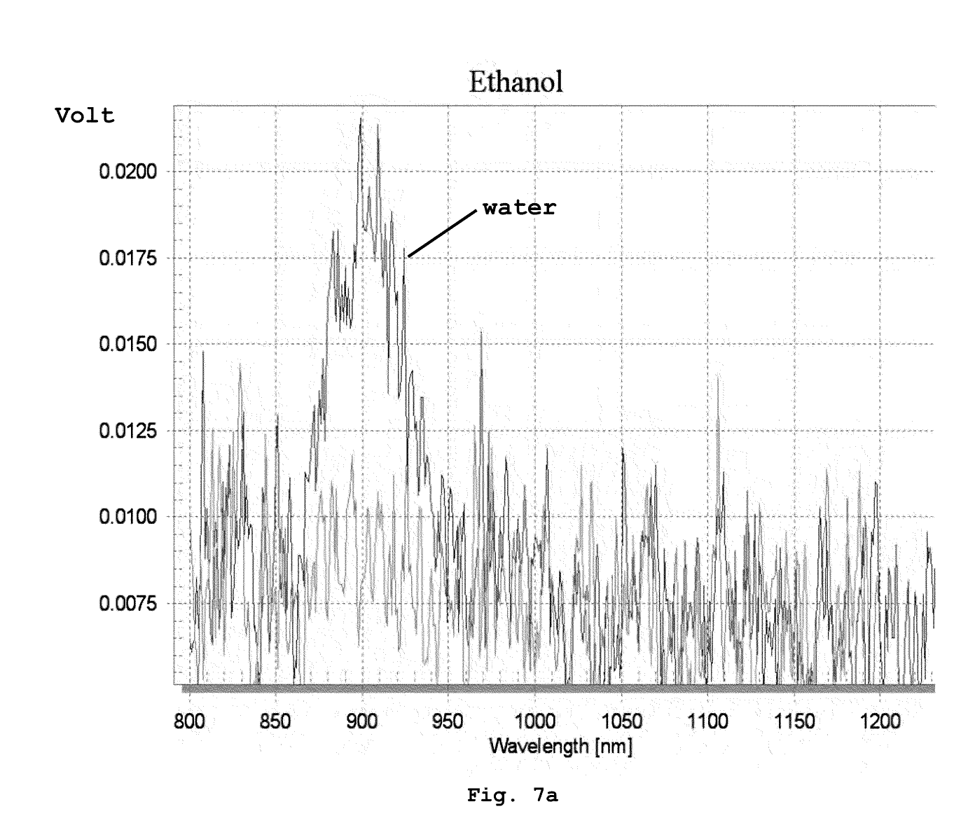

FIG. 6 illustrates the NIR fluorescence of platelets and the fluorescence of alteration in platelets induced by agonists. Spectra 1 illustrates a time scan for platelets (at 900 nm emission) in absence of an agonist while spectra 2 illustrates a time scan for platelets in the presence of an agonist (2.5 uM collagen). The magnitude of the responses in FIG. 6 provide evidence that the method can be applied to characterize microscale objects such as platelets (2-3 um). FIG. 6 also provides evidence that one can characterize the microaggregation of such objects. As platelet micro aggregate formation is important in cardiovascular disease, the method may be used to study the progress of cardiovascular disease.

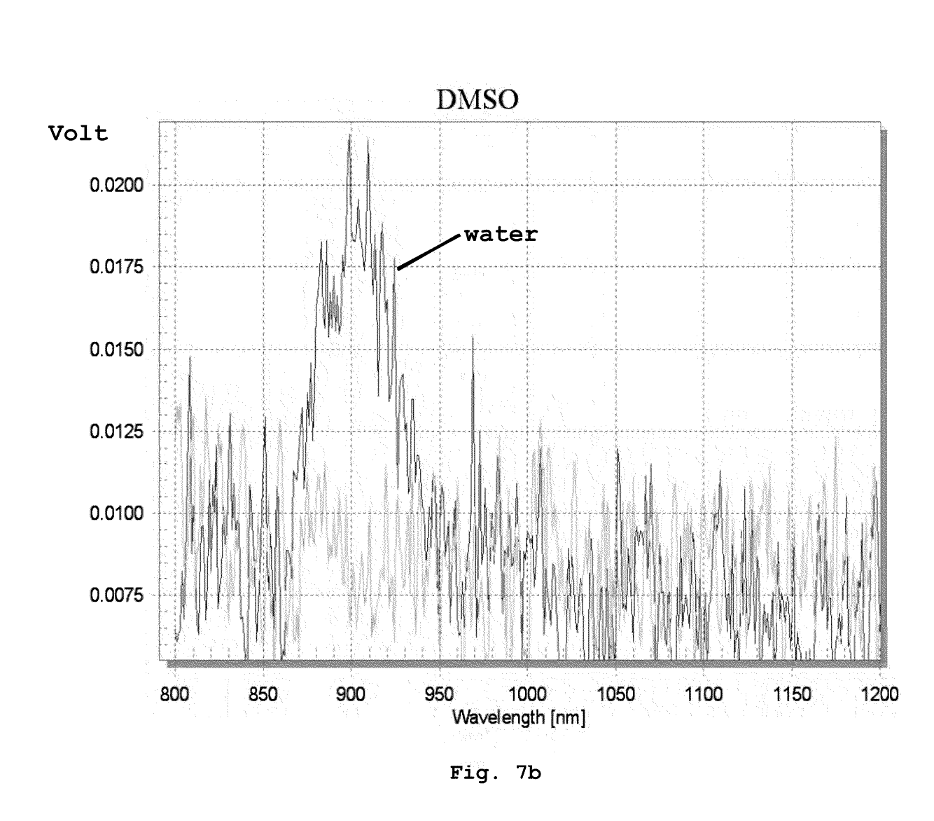

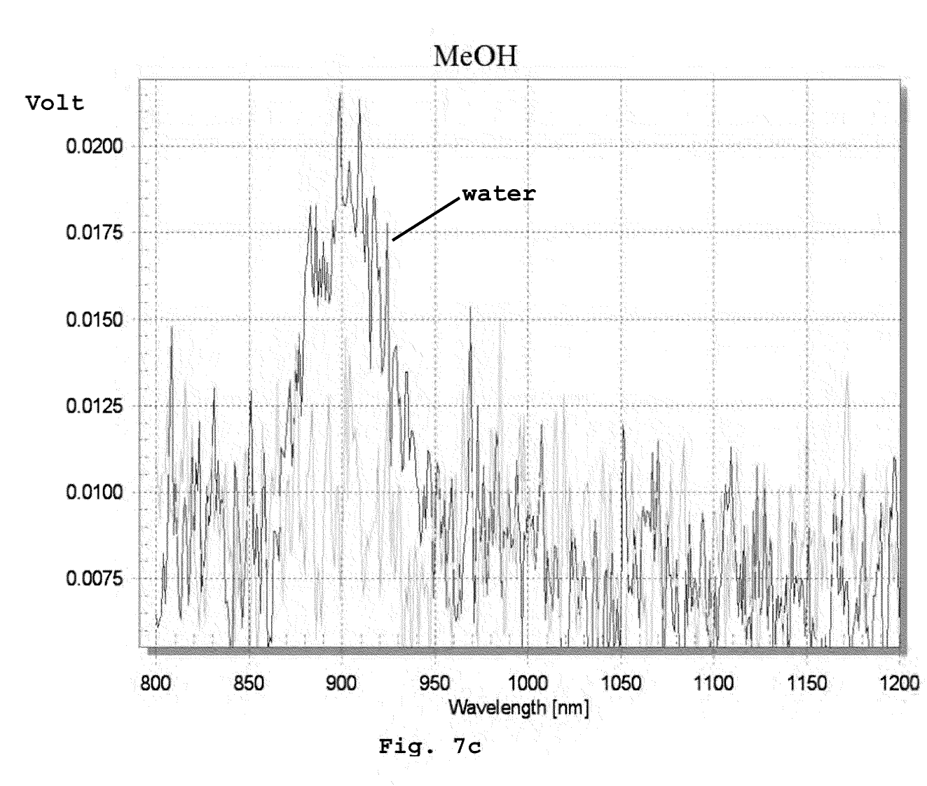

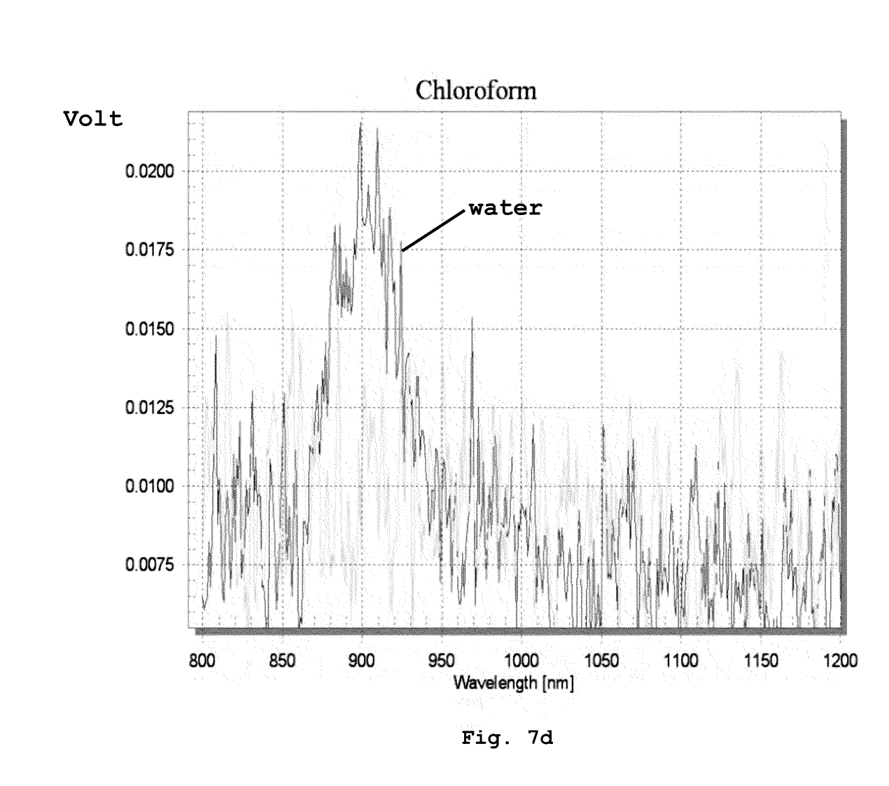

FIG. 7 illustrates the NIR fluorescence specificity of water relative to other solvents including: (a) ethanol, (b) DMSO, (c) methanol (MeOH), and (d) chloroform. For each of the four non-aqueous solvents, a water spectra has been included for comparison. As can be seen in the Figures, only water has a discernable peak in the NIR.

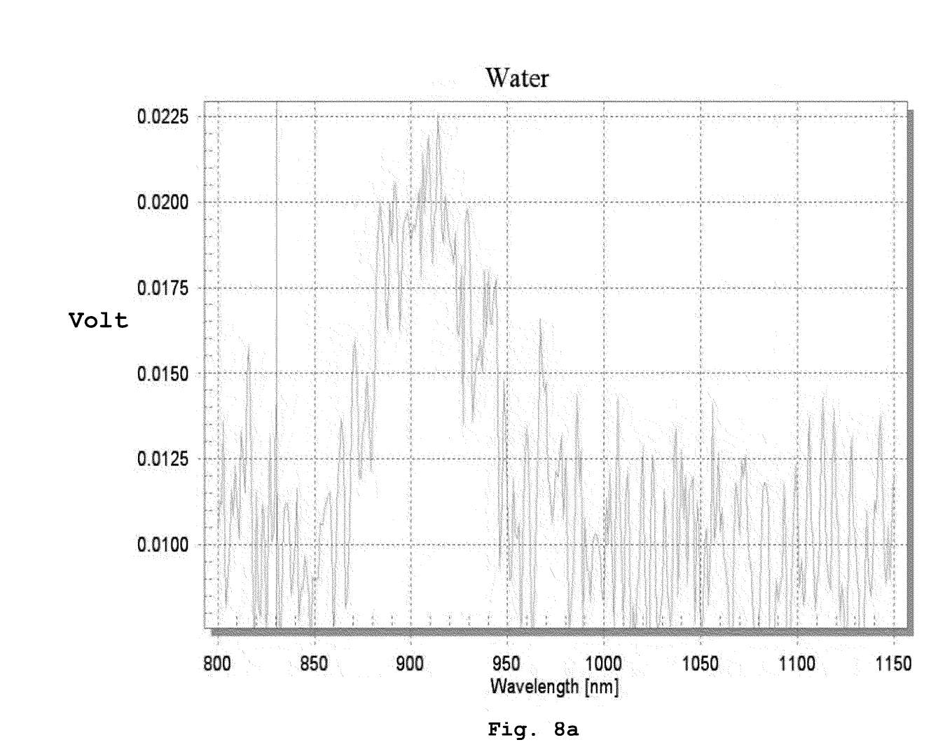

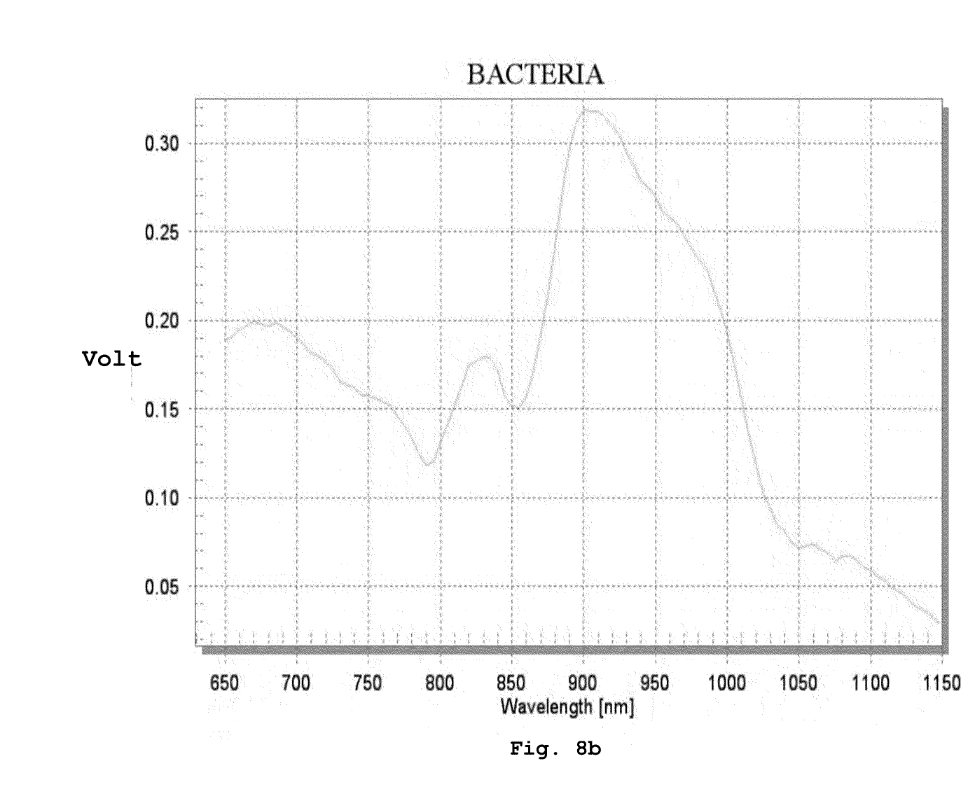

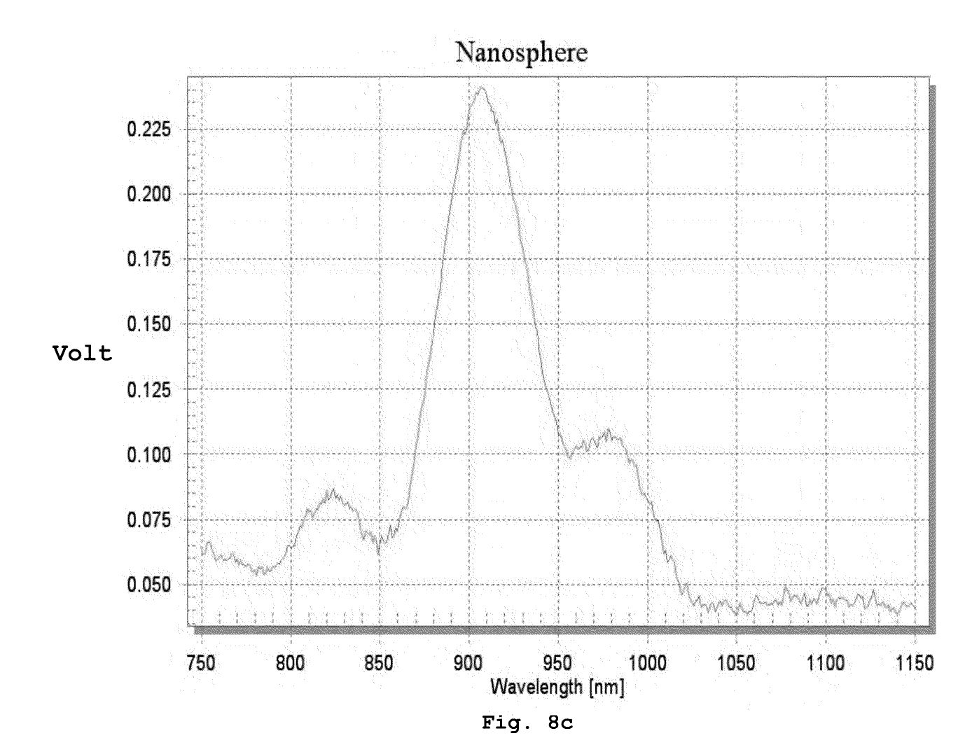

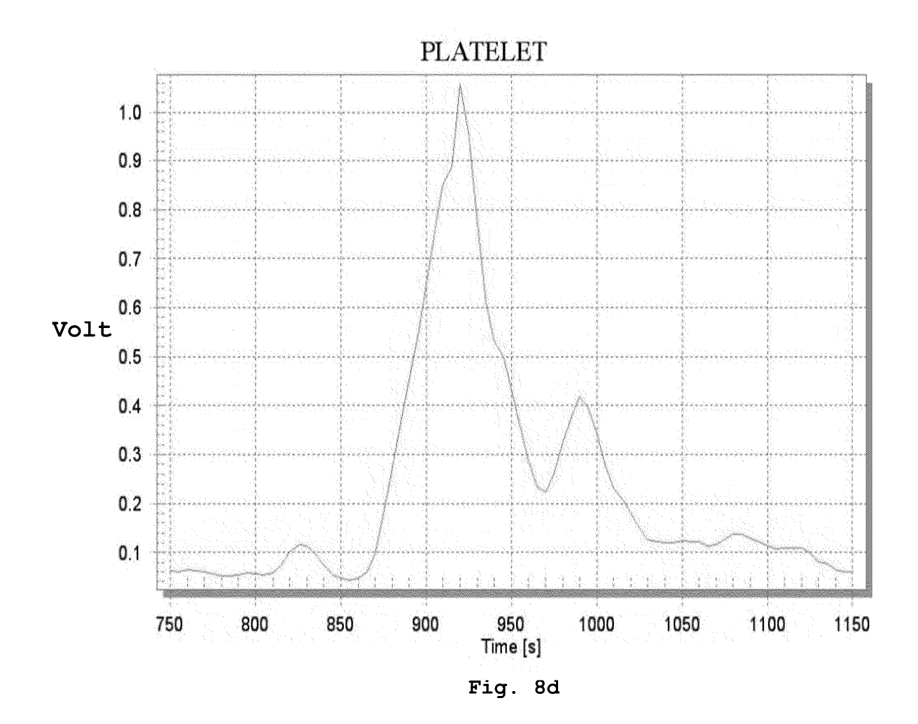

FIG. 8 illustrates NIRF diversity to different water/particle interfaces including: (a) pure water, (b) bacteria, (c) gold nanospheres, and (d) platelets. Each of the particles cause a peak having a significant increase in intensity relative to pure water at 900 nm—bacteria approximately 15×, gold nanospheres approximately 10×, platelets approximately 45×. Further, the shape of each of the peaks is different. Therefore, is it possible with this method to not only detect nano and micro particles but to also distinguish between types of particles.

FIG. 9 illustrates the hydrodynamic size of nanoparticles used in the Examples, including: (a) 20 nm gold nanopartilces (GNP), (b) 30 nm GNP, (c) 40 nm, (d) 150 nm GNP, GNP, (e) 30 nm GNP coated with arginine, (f) 40 nm GNP coated with arginine, (g) 150 nm GNP coated with arginine, and (h) 60 nm latex. As can be seen from the Figures, the size of the nanoparticles has a measurable effect on the fluorescent response (compare FIGS. 9(a)-(d)). Comparing FIGS. 9(e)-(h) with FIGS. 9(a)-(d), one can also see that the surface properties of nanoparticle also has an effect. That is, arginine coated gold nanoparticles can be distinguish from non-coated gold nanoparticles of the same size. Further, latex beads can be distinguished from gold nanoparticles.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method comprising:
exposing a sample comprising water to near infrared (NIR) light; and
detecting the presence of one or more objects by measuring a shift in an intensity of a peak in an emission spectra in the NIR,
wherein the intensity of the peak is a function of size and concentration of the one or more objects in the sample.

2. The method of claim 1, wherein the detected object is not conjugated with an NIR label.

3. The method of claim 1, wherein the method is sensitive to object size.

4. The method of claim 1, wherein the method integrates detection of nanoparticles and drug targeting without conjugated NIR dyes.

5. The method of claim 1, further comprising detecting a change in shape or change in surface property.

6. The method in claim 1, further comprising of interaction of an object with an agent that causes aggregation of the object.

7. The method of claim 6, further comprising detecting a disease or evaluating the progress of a disease, where the disease is marked by lowering or enhancing the extent of object aggregation.

8. The method of claim 1, wherein the excitation wavelength is approximately 640 nm and wherein the emission spectra has a peak in a range of approximately 850-1050 nm.

9. The method of claim 1, wherein the objects are capped with arginine.

10. The method of claim 1, wherein the objects are biological and further comprising modifying the object with an agonist.

11. A system comprising:
a light source configured to emit light onto a sample comprising water;
a detector configured to detect a shift in an emission spectra in the NIR; and
an imager configured to obtain an image of the sample.

12. The system of claim 11, wherein the light is emitted at a wavelength is approximately 640 nm and wherein the emission spectra has a peak in a range of approximately 850-1050 nm.

13. The system of claim 11, further comprising a sample excited by the light, the sample comprising objects.

14. The system of claim 13, wherein the objects are gold or latex beads.

15. The system of claim 13, wherein the objects are biological.

16. A method comprising:
supplying objects that range in size from 5 nm to 10 micron to a sample to be imaged, the sample comprising water;
exposing the sample to near infrared (NIR) light;
measuring a shift in an intensity of a peak in an emission spectra in the NIR; and
obtaining an image of the sample,
wherein the intensity of the peak is a function of size and concentration of the objects in the sample.

17. The method of claim 16, wherein obtaining an image comprises special mapping.

18. The method of claim 16, wherein obtaining an image comprises determining the structure of a cell.

19. The method of claim 16, wherein the objects are gold or latex beads.

20. A method comprising using the system of claim 11 for an application selected from the group consisting of:
detecting nano-scale or micro-scale objects in an aqueous sample;
imaging a biostructure;
detecting particulate pollution in a water sample;
detecting a drug conjugated nanoparticle; and
detecting aggregation of objects that lead to formation of larger objects.
